(19) United States
(12) Patent Application Publication
(10) Pub. No.: US 2003/0023274 A1
(43) Pub. Date: Jan. 30, 2003

(54) Automated external defibrillator and electrode package therefor

(75) Inventors: David Chesley, Chelsea, MA (US); Stuart Jang, Boston, MA (US); Craig Andrews, Mosman (AU); Jeffrey Siegel, Castine, ME (US); Thomas Swyst, Arlington, MA (US)

Correspondence Address: PIERCE ATWOOD, One Monument Square, Portland, ME, 04101, US

(73) Assignee: Active Corporation

(21) Appl. No.: 09/912,940

(22) Filed: Jul. 25, 2001

(51) Int. Cl. A61N 1/39

(52) U.S. Cl. 607/5

(57) ABSTRACT

An automated external defibrillator (AED) is provided with an electrode package mounted to an exterior surface of its casing. The electrode package includes a tray, two electrodes disposed in the tray, and a cover attached to the tray. The tray can include an outer rim that surrounds a recessed receptacle portion, with the cover being attached to the outer rim so as to cover the receptacle portion. The cover has a pull tab formed thereon and graphics that illustrate how the electrodes are accessed and used. A connector is mounted to the outer rim and has a first end that extends into the receptacle portion and a second end that extends out of the tray. The electrodes are connected to the first connector end, and the second end of the connector is inserted into a socket located on the exterior surface of the casing for electrically connecting the electrodes to the unit.

### BACKGROUND OF THE INVENTION

This invention relates generally to automated external defibrillators and more particularly to automated external defibrillators that can be easily used by people not specifically trained to use the device.

External defibrillators are commonly used to treat people suffering from cardiac arrest. Defibrillators deliver an electric pulse to the patient's heart through electrodes applied to the patient's chest. The shock tends to terminate heart arrhythmia and restore a normal rhythm. The sooner defibrillation is provided after the onset of cardiac arrest, the more likely the patient is to survive. Ideally, a cardiac arrest patient is defibrillated in less than four minutes. Beyond ten minutes, the survival rate is virtually zero.

Manual external defibrillators are normally located in hospital emergency rooms, operating rooms, and emergency medical vehicles. However, since cardiac arrest can strike unexpectedly, a person may not be anywhere near one of these devices when cardiac arrest occurs. This makes the goal of providing defibrillation in less than four minutes very difficult to meet in many instances. Furthermore, manual external defibrillators can be used by only highly trained and skilled personnel.

Automated external defibrillators (AEDs) have been developed that are portable and can be used by people without extensive training. Generally, AEDs differ from manual external defibrillators in that AEDs analyze electrocardiogram (ECG) data and provide an indication to the user of when to apply the electric shock. To resuscitate a cardiac arrest victim, an AED user applies a pair of defibrillation electrodes to the victim's chest and then operates the AED through its user interface. Automated external defibrillators are being provided in police cars, fire trucks and airplanes for use by first responders such as police officers, fire fighters and flight attendants. Because they can be used by people with little or no training, AEDs are also being deployed more and more in places where large numbers of people are often found, such as airports, shopping malls and sporting arenas.

Because AEDs are often used by non-medical professionals in stressful situations, it is desirable that they be as clear and easy to use as possible. To this end, AEDs are typically provided with a simple, easily understood user interface. Generally, once the AED is turned on, the user interface provides visual and/or audio prompts to interactively guide the user through the process. One known user interface employs a 1-2-3 step methodology in which the first step is to turn on the AED, the second step is to analyze the ECG (which is done automatically by the AED), and the third step is to produce the electric shock.

While existing user interfaces facilitate using an AED, they tend to neglect the most difficult and time consuming element of the process: attaching the electrodes to the patient's chest. In many AEDs, the electrodes are hermetically sealed in a foil pouch stored in the unit and are not electrically connected to the AED. The electrode pads are hermetically sealed in the foil pouches so that the conductive gel within them does not dry out. The user, under high stress, must locate and remove the pouch, connect the electrode connectors to the unit, tear open the pouch to remove the electrode pads, and attach the pads to the patient. Time studies have shown this part of the process to be the most time consuming aspect. Other AEDs save some time by having the electrode connectors pre-connected to the unit. However, the electrode pads are still disposed in a foil pouch that is stored within a closed compartment of the AED and are thus not visible unless the compartment is opened. This makes locating the electrode pads difficult, particularly for an inexperienced user under high stress conditions.

Accordingly, there is a need for an AED that is clearly understandable and easy to use under stressful situations and provides improved storage and access of the electrodes.

### SUMMARY OF THE INVENTION

The above-mentioned need is met by the present invention, which provides an automated external defibrillator (AED) having a casing and an electrode package mounted to an exterior surface of the casing. The electrode package includes a tray, two electrodes disposed in the tray, and a cover attached to the tray. The tray can include an outer rim that surrounds a recessed receptacle portion, with the cover being attached to the outer rim so as to cover the receptacle portion. The cover has a pull tab formed thereon and graphics that illustrate how the electrodes are accessed and used.

A connector is mounted to the outer rim and has a first end that extends into the receptacle portion and a second end that extends out of the tray. The electrodes are connected to the first connector end. The electrode package can also have a razor disposed in the tray.

The electrode package is mounted in a recess formed in the exterior surface of the casing and is retained at one end by at least one hook formed on the casing. The second end of the connector is inserted in a socket located in the exterior surface adjacent to the recess for electrically connecting the electrodes to the unit and retaining the other end of the electrode package.

The present invention and its advantages over the prior art will become apparent upon reading the following detailed description and the appended claims with reference to the accompanying drawings.

### DESCRIPTION OF THE DRAWINGS

The subject matter that is regarded as the invention is particularly pointed out and distinctly claimed in the concluding part of the specification. The invention, however, may be best understood by reference to the following description taken in conjunction with the accompanying drawing figures in which:

FIG. 1 is a top view of an automated external defibrillator (AED) having a top-mounted electrode package.

FIG. 2 is a perspective view of the AED showing a second electrode package partially removed.

FIG. 3 is a front view of the AED.

FIG. 4 is an exploded view of the electrode package.

FIG. 5 is a top view of the AED with the electrode package cover omitted to reveal the contents of the electrode package.

FIG. 6 is a top view of the AED with the electrode package omitted to reveal the underlying structure.

FIG. 7 is a partial cross-section view taken along line 7-7 of FIG. 1 to show detail of the electrode package mounting arrangement.

### DETAILED DESCRIPTION OF THE INVENTION

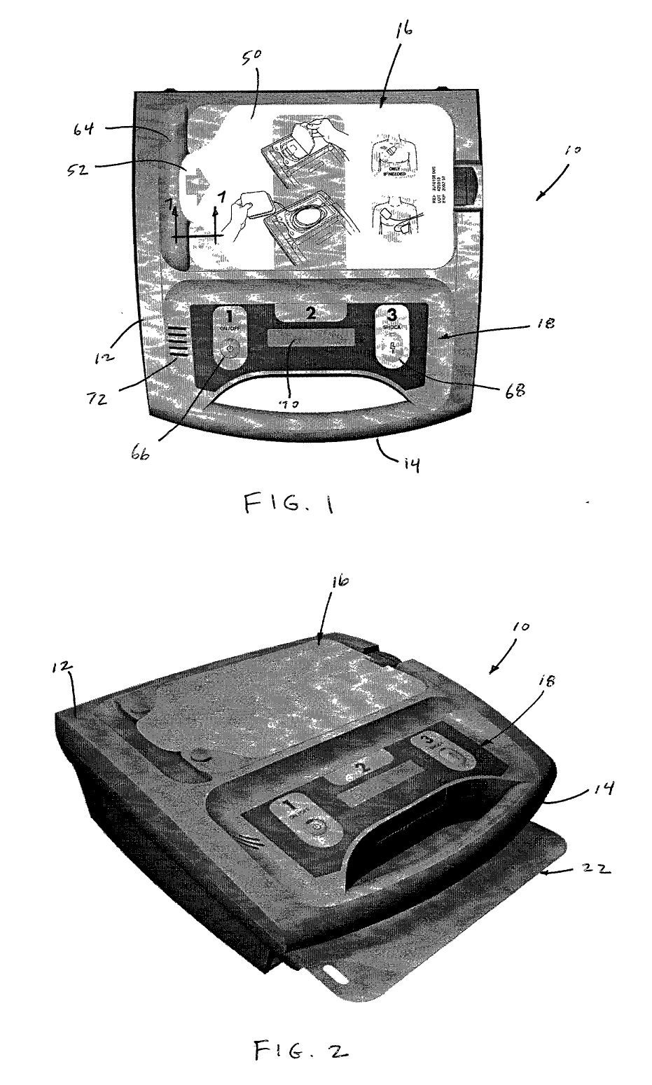

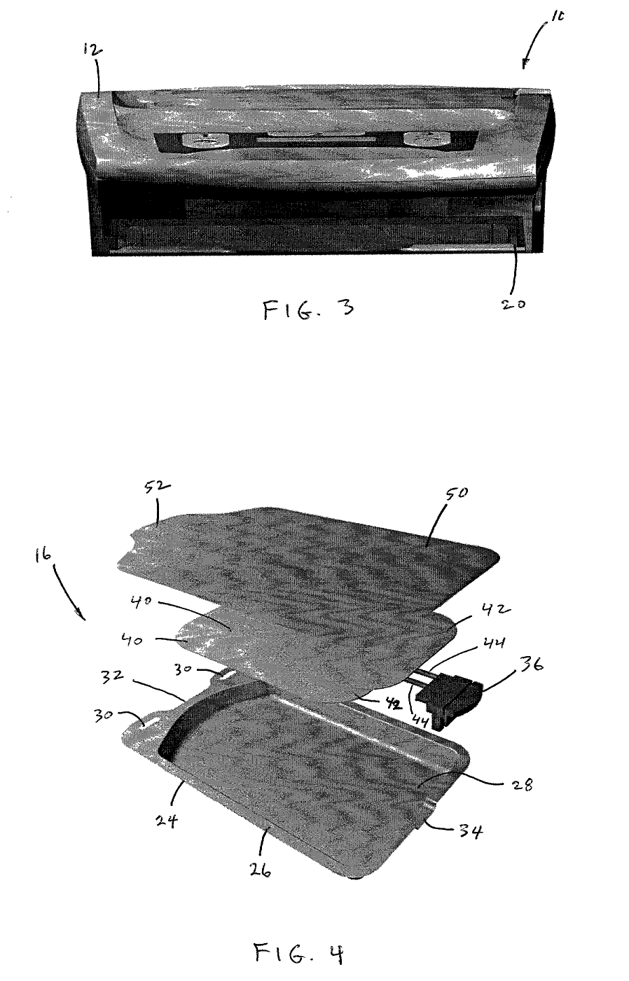

Referring to the drawings wherein identical reference numerals denote the same elements throughout the various views, FIGS. 1-3 show an automated external defibrillator (AED) 10 in accordance with one embodiment of the invention. The AED 10 includes an outer casing 12 that houses conventional defibrillation electronics and has a handle 14 formed on the front end thereof. An electrode package 16 is mounted to the exterior of the casing 12, on the top surface thereof. A user interface section 18 is also located on the top surface of the casing 12, between the handle 14 and the electrode package 16. As best seen in FIG. 3, the AED 10 further includes an internal storage compartment 20 for storing a second electrode package 22, which is identical to the first electrode package 16 in form and content. The storage compartment 20 is accessible through an opening in the front end of the casing 12, below the handle 14. The storage compartment 20 is provided with a mechanism that normally holds the second electrode package 22 in the compartment 20 but allows the second electrode package 22 to be withdrawn by a slight tug thereon. As will be described in more detail below, the second electrode package 22 serves as a backup or replacement for the primary electrode package 16.

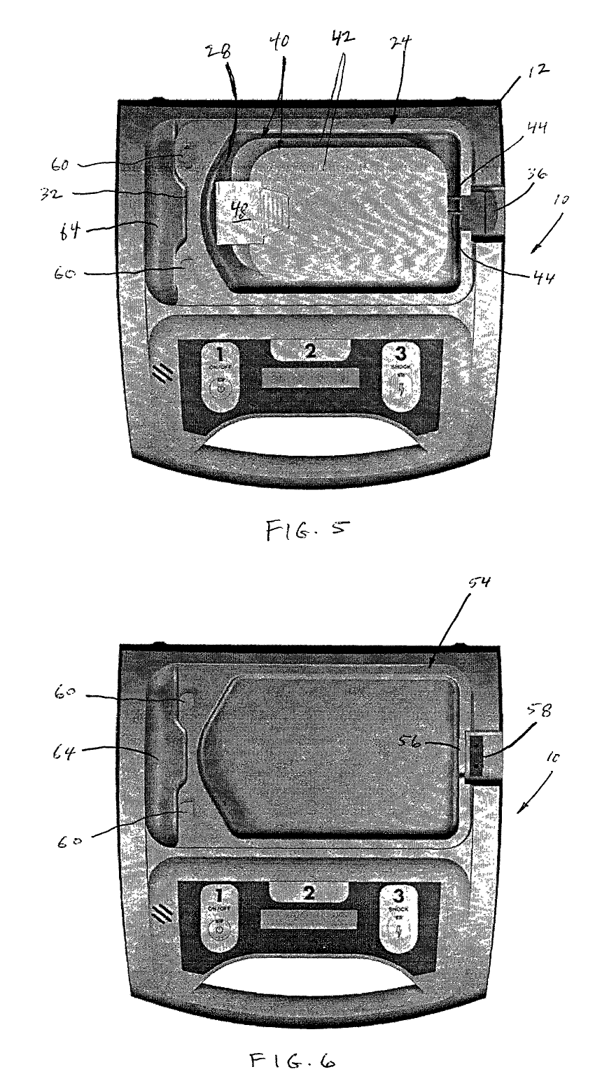

Turning to FIGS. 4 and 5, the electrode package 16 includes a rectangular tray 24 that is preferably, but not necessarily, made of a plastic material. The tray 24 has an outer rim 26 that surrounds a recessed receptacle portion 28. The outer rim 26 is larger at a first end of the tray 24. This enlarged portion of the rim 26 has two openings 30 and a slot 32 formed therein. A notch 34 is formed in the outer rim 26, at a second end of the tray 24 that is opposite the first end.

The electrode package 16 further includes a connector 36 that fits into the notch 34 of the tray 24. When properly positioned in the notch 34, the connector 36 has a first end that extends into the receptacle portion 28 and a second end that extends out of the tray 24. The second end of the connector 36 is provided with two coupling elements, the purpose of which is described below.

A pair of electrodes 40 is stored in the receptacle portion 28. Each electrode 40 includes an electrode pad 42 and a cable 44 connected at one end to the electrode pad 42. The other end of each cable 44 is connected to the first end of the connector 36. The cables 44, which are long enough to extend from the AED 10 to the patient's chest, are coiled and stowed under the electrode pads 42. As is generally known in the art, each electrode pad 42 comprises an electrode element that is electrically connected to the cable 44. A conductive gel is provided for ensuring good electrical conductivity between the electrode element and the patient's body. Each electrode pad 42 preferably includes an adhesive material for adhering the electrode pad 42 to the patient's chest. A releasable backing sheet is disposed over the adhesive to prevent the electrode pad from unintentionally adhering to objects while not in use.

A razor 48 is optionally stored in the receptacle portion 28. The razor 48, which can simply be a commercially available disposable razor, is provided to remove chest hair if needed. Thick chest hair can interfere with the quality of the electrical connection between the patient and the electrode pad 42. Thus, if the patient has thick chest hair, the razor 48 can be used to quickly shave the areas of the chest to which the electrode pads 42 are to be attached.

A cover 50, which is preferably a foil or plastic film such as a Mylar film, is releasably attached to the rim 26 of the tray 24 so as to cover the receptacle portion 28. The cover 50 is generally rectangular in shape and defines an outer edge that matches the outer edge defined by the rim 26 on three sides thereof. The fourth side of the cover 50, which side corresponds to the end of the tray 24 having the enlarged portion of the rim 26, includes a pull tab 52 designed to be grasped by a user. With the cover 50 in place on the tray 24, the tab 52 overlays the slot 32 in the rim 26 and extends beyond the edge of the tray 24. The pull tab 52 has a sufficiently narrow width so as to leave the two openings 30 uncovered. The cover 50 also overlays the connector 36 located in the notch 34.

The cover 50 is a “peel-away” cover; that is, it is attached to the rim 26 by an adhesive that securely retains the cover 50 but allows it to be peeled off when the tab 52 is pulled. Any suitable releasable adhesive can be used for this purpose. When so attached, the cover 50 hermetically seals the receptacle portion 28 so as to generally protect the contents thereof and prevent the conductive gel of the electrodes 40 from drying out.

The cover 50 can also be provided with graphics that illustrate how the electrodes 40 are to be used for the defibrillation process. For example, FIG. 1 shows graphics that illustrate 1) opening the electrode package 16 by peeling off the cover 50, 2) the contents of the electrode package 16 and the electrode pads 42 being removed by hand, 3) that the razor is used to shave off chest hair if needed, and 4) where on the patient to attach the electrode pads 42. Thus, with a quick glance at the cover graphics, a user can easily deduce how to access and use the electrodes 40.

Completely assembled, the electrode package 16 comprises the electrodes 40 and the razor 48 hermetically sealed in the receptacle portion 28 of the tray 24 by the cover 50. The connector 36 is snapped in place in the notch 34. The second electrode package 22 is structurally identical to the first electrode package 16 and differs only in the manner in which it is mounted to the casing 12.

Referring now to FIG. 6, a recess 54 is formed in the top surface of the casing 12 and is sized and shaped to receive the electrode package 16. The recess 54 includes a deep portion that receives the receptacle portion 28 and a shallow portion that surrounds the deep portion and receives the rim 26. An indentation 56 is formed in the shallow portion at one end of the recess 54 for receiving the notch 34 of the rim 26. An electrical socket 58 is located in the top surface of the casing 12, adjacent to the indentation 56. When the electrode package 16 is positioned in the recess 54, the connector 36 is inserted into the socket 58. Specifically, the coupling elements of the connector 36 engage mating coupling elements in the socket 58 to form an electrical connection. The socket 58 is electrically connected to the AED electronics located inside the casing 12. Thus, the electrode pads 42 are electrically connected to the AED electronics when the connector 36 is inserted into the socket 58. As is known in the art, the AED electronics analyze electrocardiogram (ECG) data input from the electrodes 40 and provide an indication via the user interface section 18 of when to apply an electric shock. The electronics can be any type of control system suitable for use in AEDs, many of which are known, and are thus not described in detail here.

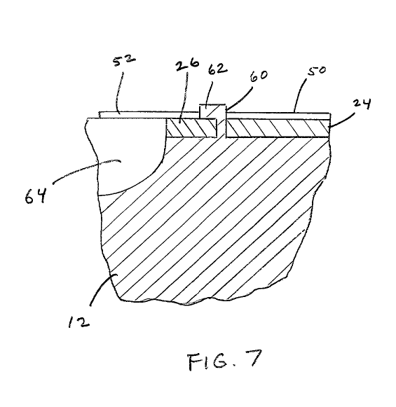

Two hooks 60 are integrally formed on the casing 12 in the shallow portion of the recess 54. The hooks 60, which are located near the end of the recess 54 that is opposite from the socket 58, are sized and positioned to engage the openings 30 formed in the rim 26. Referring to FIG. 7, each hook 60 has a lip 62 that extends away from the deep portion of the recess 54. The recess 54 further includes a second deep portion 64 located at the end of the recess 54 opposite the socket 58. The section of the shallow portion that supports the hooks 60 is disposed between the second deep portion 64 and the first deep portion of the recess 54. The second deep portion 64 provides an open space under the pull tab 52 to facilitate grasping the tab 52.

To mount the electrode package 16 to the casing 12, the openings 30 are placed over the hooks 60. The electrode package 16 is then slid across the casing 12, toward the socket end of the recess 54. Respective portions of the rim 26 adjacent each opening 30 become disposed under the lips 62, as shown in FIG. 7, and the pull tab 52 overlies the second deep recess portion 64. The hooks 60 retain that end of the electrode package 16 to the casing 12, even while the cover 50 is being pulled off. The connector 36 is then snapped into the socket 58 for retaining the other end of the electrode package 16 to the casing 12 as well as electrically connecting the electrodes 40. With this configuration, mounting the electrode package 16 to the casing 12 automatically connects the electrodes 40 to the AED electronics; the electrode package 16 cannot be completely seated in the recess 54 unless the connector 36 is snapped into the socket 58.

Referring again to FIG. 1, the user interface section 18 includes an on/off button 66 for turning the AED 10 on or off and a shock button 68 that will cause the AED 10 to deliver an electric shock to the patient when pressed. A display 70 is included for providing visual prompts and instructions to interactively guide a user through a defibrillation process. The display 70 can be an LCD display, an LED display or any other suitable alpha-numeric display. A speaker 72 is included for providing audio prompts in addition to the visual prompts provided by the display 70. Once the AED 10 is turned on, the display 70 and speaker 72 are controlled by the AED electronics in a manner known in the art.

The user interface section 18 also includes labeling for employing a 1-2-3 step methodology. Specifically, the on/off button 66 is labeled “1” as an indication that turning the AED 10 on is the first step. The electrode package 16 is labeled “2” to indicate that accessing and deploying the electrodes 40 is the next step. The shock button 68 is labeled “3” as an indication that producing a shock is the last step. The 1-2-3 step methodology of the present invention differs from prior art approaches in that attaching the electrodes 40 to the patient (which can be the most difficult and time consuming part of the process) is identified as the second step. Prior AEDs that use 1-2-3 step methodology typically identify analyzing the ECG data as the second step. However, this approach is not particularly helpful to a user because the ECG analysis is done automatically by the AED and does not require specific action by the user.

In operation, the user first activates the AED 10 by pressing the on/off button 66. This begins the AED 10 and the display 70 and/or speaker 72 will prompt the user to the next step, which is attachment of the electrodes 40. As led by the graphics on the cover 50, and possibly instructions from the display 70 and/or speaker 72, the user will peel off the cover 50 from the electrode package 16 and remove the razor 48 and electrodes 40. If necessary, the user will use the razor 48 to shave chest hair from the patient. The user then removes the releasable backing sheets from the electrode pads 42 and adheres the electrode pads 42 to the patient at the locations shown by the graphics on the cover 50. If either of the electrode pads 42 accidentally folds over such that two portions thereof become adhered to one another, the pad becomes essentially unusable. In this case, the user can completely remove the opened electrode package 16 from the casing 12, remove the second electrode package 22 from the compartment 20, and then mount the second electrode package to the casing 12 for use as a backup. If not used as a backup, the second electrode package 22 would still be available as a replacement for the first electrode package 16 after a successful defibrillation process.

The electrode pads are already connected to the AED electronics via the cables 44, the connector 36 and the socket 58. Once the electrode pads 42 are attached to the patient, the AED electronics will begin analyzing ECG data being received by the electrodes 40. When and if the AED electronics detect a shockable rhythm, the user interface will prompt the user to initiate a shock by pressing the shock button 68. This prompt can be accomplished visually via the display 70 and/or audibly via the speaker 72. The shock button 68 can also be illuminated to assist the user. When the user presses the shock button 68, the electric shock is produced and delivered through the electrodes 40. The AED 10 then continues to monitor the ECG data for determining whether another shock is necessary.

The AED 10 provides many advantages over prior devices. For example, by mounting the electrode package 16 to the exterior of the casing 12, the present invention utilizes a “billboarding” concept. That is, the electrode package 16 is clearly visible to a user as opposed to being hidden inside the casing 12. This greatly facilitates locating the electrodes 40, which was not always a simple step in prior devices, and the peel-away cover 50 simplifies the step of accessing the electrodes. Furthermore, the graphics on the cover 50 make it clear, even to an inexperienced user, how the electrodes 40 are accessed and attached to the patient. Another advantage of the billboarding concept is that the electrode package 16 and the entire user interface are always exposed. Automated external defibrillators that are deployed in public places such as airports, shopping malls and sporting arenas are only taken out when they need to be used. It is only then that a user needs to figure out how to use the electrodes. With billboarding, the electrode package, cover graphics and user interface are always visible. This allows a potential future user to see the entire set up every time he or she walks by the AED. Accordingly, such passersby will tend to develop an understanding of how the AED 10 works (even if only on a subliminal level) before the need to use the device arises.

Yet another advantage of the top-mounted electrode package involves preparing the AED 10 for another use after it has been used for a defibrillation procedure. The AED 10 can be prepared for subsequent use by mounting another electrode package (either the second electrode package 22 or a new electrode package) to the casing 12. Other AEDs can also have new electrodes provided. However, with the top-mounted electrode package, it would be quite clear that the AED 10 is not ready if an electrode package has not been reloaded. With prior AEDs in which the electrodes are disposed in an internal compartment, it would be possible to mistakenly think that electrodes had been reloaded when in fact they had not. The AED 10 also provides the benefit of a backup electrode package 22 that can be used to provide a second chance in the event some element of the primary electrode package becomes damaged.

While specific embodiments of the present invention have been described, it will be apparent to those skilled in the art that various modifications thereto can be made without departing from the spirit and scope of the invention as defined in the appended claims.

What is claimed is:

1. An automated external defibrillator comprising:
a casing; and
an electrode package mounted to an exterior surface of said casing, said electrode package including a tray, two electrodes disposed in said tray, and a cover attached to said tray.

2. The automated external defibrillator of claim 1 wherein said exterior surface is a top exterior surface of said casing.

3. The automated external defibrillator of claim 1 further comprising an internal compartment formed in said casing for receiving an electrode package.

4. The automated external defibrillator of claim 3 further comprising a second electrode package disposed in said internal compartment, said second electrode package including a tray, two electrodes disposed in said tray, and a cover attached to said tray of said second electrode package.

5. The automated external defibrillator of claim 1 further comprising a recess formed in said exterior surface for receiving said electrode package.

6. The automated external defibrillator of claim 5 wherein said tray includes an outer rim that surrounds a recessed receptacle portion, said cover being attached to said outer rim so as to cover said receptacle portion.

7. The automated external defibrillator of claim 6 wherein said recess includes a deep portion that receives said receptacle portion and a shallow portion that receives said outer rim.

8. The automated external defibrillator of claim 6 further comprising a connector mounted to said outer rim, said connector having a first end that extends into said receptacle portion and a second end that extends out of said tray.

9. The automated external defibrillator of claim 8 wherein said electrodes are connected to said first end of said connector.

10. The automated external defibrillator of claim 8 further comprising a socket located in said exterior surface adjacent to said recess, said second end of said connector being inserted into said socket.

11. The automated external defibrillator of claim 6 further comprising at least one hook formed on said casing in said shallow portion of said recess and at least one opening formed in said outer rim, wherein said hook engages said opening.

12. The automated external defibrillator of claim 11 wherein said cover is a peel-away cover releasably attached to said tray, said peel-away cover having a pull tab formed on a first end thereof, said first end being adjacent to said opening.

13. The automated external defibrillator of claim 1 wherein said cover has graphics printed thereon that illustrate how said electrodes are accessed and used.

14. The automated external defibrillator of claim 1 further comprising a razor disposed in said tray.

15. An electrode package for use in automated external defibrillators, said electrode package comprising:
a tray;
two electrodes disposed in said tray; and
a cover attached to said tray.

16. The electrode package of claim 15 wherein said tray includes an outer rim that surrounds a recessed receptacle portion, said cover being attached to said outer rim so as to cover said receptacle portion.

17. The electrode package of claim 16 further comprising a connector mounted to said outer rim, said connector having a first end that extends into said receptacle portion and a second end that extends out of said tray.

18. The electrode package of claim 17 wherein said electrodes are connected to said first end of said connector.

19. The electrode package of claim 15 further comprising a razor disposed in said tray.

20. The electrode package of claim 15 wherein said cover is a peel-away cover releasably attached to said tray.

21. The electrode package of claim 20 wherein said peel-away cover has a pull tab formed on one end thereof.

22. The electrode package of claim 15 wherein said cover has graphics printed thereon that illustrate how said electrodes are accessed and used.
